(19) United States
(12) Patent Application Publication
(10) Pub. No.: US 2021/0346304 A1
(43) Pub. Date: Nov. 11, 2021

(54) ENCAPSULATED CELLS PRODUCING CYTOCHROME P450 AND METHODS OF USE THEREOF

(71) Applicant: PharmaCyte Biotech, Inc., Laguna Hills, CA (US)

(72) Inventors: Kenneth L. WAGGONER, Lake Forest, CA (US); Gerald W. CRABTREE, Groton, CT (US)

(73) Assignee: PharmaCyte Biotech, Inc., Laguna Hills, CA (US)

(21) Appl. No.: 17/123,941

(22) Filed: Dec. 16, 2020

Related U.S. Application Data
(63) Continuation of application No. 15/927,528, filed on Mar. 21, 2018, now abandoned.
(60) Provisional application No. 62/474,463, filed on Mar. 21, 2017.

(51) Int. Cl. A61K 9/50; A61K 9/00; A61K 38/41; A61P 35/00; C12N 9/02

(52) U.S. Cl. CPC A61K 9/5042; A61K 9/0056; A61K 31/4164; A61P 35/00; C12N 9/0077; A61K 38/415

(57) ABSTRACT

Disclosed herein are methods and compositions to treat solid tumors. In one embodiment, the method of treating a solid tumor in a patient includes administering at the tumor site a therapeutically effective amount of cytochrome P450 producing cells encapsulated in a capsule and administering a prodrug which is activated by cytochrome P450, wherein the prodrug is administered at least three or more cycles, and wherein each cycle comprises three consecutive daily administration.

### CLAIM OF PRIORITY

This application is a Continuation of U.S. Non-Provisional application Ser. No. 15/927,528 filed Mar. 21, 2018, titled “ENCAPSULATED CELLS PRODUCING CYTOCHROME P450 AND METHODS OF USE THEREOF”, which claims priority to U.S. Provisional Application No. 62/474,463 filed on Mar. 21, 2017, titled “ENCAPSULATED CELLS PRODUCING CYTOCHROME P450 AND METHODS OF USE THEREOF”, each of which are hereby incorporated by reference.

Disclosed herein are compositions and methods for treating cancer using such compositions. In one embodiment, a method of treating a solid tumor in a subject includes administering at the tumor site a therapeutically effective amount of cytochrome P450 producing cells encapsulated in a capsule and administering a prodrug which is activated by cytochrome P450, wherein the prodrug is administered at least three or more cycles, and wherein each cycle comprises three consecutive daily administrations. In an additional embodiment, the capsule may be permeable to the prodrug and the prodrug is converted into an active moiety by cytochrome P450. In a further embodiment, the prodrug may be administered at a daily dose of about 1 gm/m²/day to about 2 gm/m²/day. The period between two cycles of the prodrug administration is at least 18 days, and the prodrug administration dose may be about 3 cycles to 12 cycles. In another embodiment, the cytochrome P450 producing cells and the prodrug are administered together or sequentially, and the prodrug is administered systemically, locally, subcutaneously, intravenously, intramuscularly, intraperitoneally, transdermally, or orally. In some embodiments, the cytochrome P450 producing cells are HEK293 cells stably expressing cytochrome P450.

In some embodiments, a method of treating a subject suffering from pancreas cancer that is no longer responsive to combination of gemcitabine and nab-paclitaxel comprises administering at or near the tumor site a therapeutically effective amount of cytochrome P450 producing cells encapsulated in a capsule, and administering a prodrug which is activated by cytochrome P450.

In some embodiments, the method of treating a subject suffering from pancreas cancer that is resistant to combination of gemcitabine and nab-paclitaxel comprises administering at or near the tumor site a therapeutically effective amount of cytochrome P450 producing cells encapsulated in a capsule and administering a prodrug which is activated by cytochrome P450, wherein the prodrug is administered at least three or more cycles, and wherein each cycle comprises three consecutive daily administrations.

In another embodiment, a syringe includes a plurality of capsules, each capsule comprising a plurality of cytochrome P450 producing cells, and wherein the capsule membrane is permeable to a prodrug. In some embodiments, the cytochrome P450 producing cells are HEK293 cells. Further, the capsule membrane is made from sulfate group containing polysaccharides, polysaccharide derivatives, sulfonate group containing synthetic polymers, polymers with quaternary ammonium groups, and combinations thereof. In some embodiments, the capsule has an average diameter of about 0.001 mm to about 5 mm. In addition, the capsule membrane is porous and has a pore size from about 80 nm to about 150 nm. Further, the capsule may include about 100-10 million HEK293 cells stably expressing cytochrome P450 gene. The capsule membrane is permeable to prodrug, such as oxazaphosphorines (ifosfamide and ifosfamide products or products of other oxazaphosphorines) that have been metabolized by cytochrome P450 protein. In some embodiments, inner walls of the syringe is made of a cell non-adhesive material. Further, the syringe may also include a membrane that is permeable to oxygen and CO₂.

### BRIEF DESCRIPTION OF THE FIGURES

FIG. 1 displays Kaplan-Meier survival curves of the two clinical studies in patients with advanced pancreas cancer conducted with CypCaps and two different doses of ifosfamide treatment, according to an embodiment. CypCaps are capsules containing cells that produce cytochrome P450. The FIGURE shows Kaplan-Meier curves describing the survival of patients from the Phase 1/2 clinical studies (dotted line), the Phase 2 clinical study (dashed line) and an age and disease-stage matched historic control group receiving the best available standard care (solid line).

### DETAILED DESCRIPTION

As used in this application and in the claims, the singular forms “a,” “an,” and “the” include the plural forms unless the context clearly dictates otherwise. Additionally, the term “includes” means “comprises.”

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” indicates mammals, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

A “therapeutically effective amount” of a composition is a predetermined amount calculated to achieve the desired effect, i.e., to ameliorate, prevent or improve an unwanted condition, disease or symptom of a patient. The activity contemplated by the present methods may include both therapeutic and/or prophylactic treatment, as appropriate. The specific dose of the agent administered according to this invention is to obtain therapeutic and/or prophylactic effects. These will, of course, be determined by the particular circumstances surrounding the case, including, for example, the compound administered, the route of administration, and the condition being treated. The effective amount administered may be determined by a physician in the light of the relevant circumstances, including the condition to be treated, the choice of the effective agent to be administered, and the chosen route of administration.

As used herein, the phrase “treating solid tumor” refers to inhibition of cancer cell replication, apoptosis, inhibition of cancer spread (metastasis), inhibition of tumor growth, reduction of cancer cell number or tumor growth, decrease in the malignant grade of a cancer (e.g., increased differentiation), or improved cancer-related symptoms.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

Many malignant tumors do not respond well to chemotherapy. The anti-cancer drugs used to treat tumors are in most cases applied systemically and therefore spread through the whole body of the patient. The high systemic dose of such drugs required for cancer treatment often carries with it unpleasant side-effects for the patient. One strategy by which these problems of high systemic concentration of anti-cancer drugs could be circumvented is by the direct application or by the activation of the drug directly into or near the tumor. This could be achieved by implantation of encapsulated cells producing an activating enzyme, for example cytochrome P450, capable of converting a prodrug into an active moiety near tumor cells. The release of the active moiety of the drug from the capsules leads to high local concentrations of the activated toxic metabolites of the drug. This high local concentration of the toxic metabolites affects surrounding tumor cells in a concentration gradient dependent manner without further systemic toxicity for the patient. Such methods may be used to treat a subject with low doses of chemotherapy.

Disclosed herein are methods and compositions to treat a solid tumor in a patient. In some embodiments, a method of treating a solid tumor in a patient comprises administering at the tumor site a therapeutically effective amount of cytochrome P450 producing cells encapsulated in a capsule, and administering a prodrug which is activated by cytochrome P450, wherein the prodrug is administered at least three or more cycles, and wherein each cycle comprises three consecutive daily administrations.

In some embodiments, the cytochrome P450 producing cells are encapsulated in a capsule. The cytochrome P450 producing cells may be any mammalian cell line, such as HEK 293 cells (clone 22P1G) or CBT 4B10 cells. Introduction of the cytochrome P450 gene into the host cell can be accomplished by calcium phosphate transfection, DEAE-dextran mediated transfection, cationic lipid-mediated transfection, electroporation, transduction, infection, or other known methods. Such methods are described in many standard laboratory manuals, such as Sambrook et al., Molecular Cloning, A Laboratory Manual. 3^{rd} ed. Cold Spring Harbor Laboratory Press (2001). The cells may stably express cytochrome P450 gene. The expression construct may be stably integrated into the genomic DNA of the cell. For example, the cells may express the cytochrome P450 gene from a constitutively active promoter, such as the cytomegalovirus promoter. In some embodiments, the cytochrome P450 gene is under transcriptional control of a target cell specific regulatory element or promoter, and/or an X-ray inducible promoter. In some embodiments, the cytochrome P450 producing cells express the 2B1 isoform of the cytochrome P450 exogenously.

In some embodiments, the cytochrome P450 producing cells are monoclonal cells or cells of a single subtype. In some embodiments, the cytochrome P450 cells are polyclonal cells or a mixture of subtypes. In some embodiments, polyclonal cells of HEK 293 may be used. In some embodiments, the cytochrome P450 producing cells are polyclonal HEK 293 cells (clone 22P1G). In some embodiments, the cytochrome P450 producing cells are HEK 293 cells (clone 22P1G) expressing CYP450 2B1 isoform exogenously. In some embodiments, the cytochrome P450 producing cells express CYP450 2B6 and CYP450 3A5 isoforms. In some embodiments, it is believed that polyclonal cells function better than monoclonal cells, when encapsulated.

Cells may be engineered to express cytochrome P450 using a typical expression vector under the transcriptional control of a strong constitutive promoter, such as the CMV promoter. Alternatively, an inducible or an X-ray dependent promoter can be used for expression of the cytochrome P450 gene. The expression vectors are transfected into cells, according to standard protocols and, subsequently, populations or clones of transduced cells can be selected.

In some embodiments, the capsule is permeable to the prodrug and the prodrug is converted into an active moiety by cytochrome P450. The capsule has a porous permeable membrane which allows nutrients to enter and waste products to leave. The pores also allow small molecules, such as the prodrug, to enter and leave the capsules. However, the pores are small enough to keep the immune system cells out, thereby protecting the living cells inside. Thus, cytochrome P450 producing cells inside the capsules are protected from cells of the host immune system and thus induce no significant immune reaction. Due to the immune protection by the capsules the present method therefore provides the possibility to encapsulate any allogenic or xenogenic cells that are transfected with an expression vector encoding the cytochrome P450 gene.

In some embodiments, the capsule has a permeable membrane made from sulfate group containing polysaccharides, polysaccharide derivatives, sulfonate group containing synthetic polymers, polymers with quaternary ammonium groups, and combinations thereof. In some embodiments, the polysaccharide derivative is polymeric sodium cellulose sulfate, cellulose acetate sulfate, carboxymethycellulose sulfate, dextran sulfate or starch sulfate. Further, non-limiting examples of quaternary ammonium groups are polydimethyldiallylammonium or polyvinylbenzyl-trimethylammonium.

Capsules encapsulating cytochrome P450 producing cells may be produced by any techniques known in the art and are also described in U.S. Pat. Nos. 6,776,985 and 6,893,634, which are incorporated herein by reference. Briefly, capsules are prepared by suspending the cells producing cytochrome P450 in a solution containing 0.5-50%, sodium cellulose sulfate and 5% fetal calf serum optionally in buffer. This suspension is then dropped by a dispensing system (e.g., air-jet system or piezoelectric system) while stirring into a precipitation bath containing 0.5-50%, polydimethyl-diallylammonium chloride, optionally in buffer. Capsule formation occurs within milliseconds. The capsules containing cells are kept in the precipitation bath for 30 seconds to 5 minutes and then washed. The rapidity of this method ensures that the cells are not unduly stressed during the entire procedure. The integrity of empty and cell filled capsules can be determined by flushing through a 19G needle with a 1 ml standard syringe and observing the capsules under phase contrast and electron microscopy.

In some embodiments, the capsules have an average diameter between 0.001 mm and 5 mm, between 0.001 mm and 1 mm, between 0.001 mm and 0.1 mm, between 0.001 mm and 0.01 mm, but preferably between 0.1 and 1 mm. Consequently, capsules can be made to contain a variable number of cells. In some embodiments, the capsule may contain about 100 cells, about 1000 cells, about 10,000 cells, about 100,000 cells, about 1 million cells, or about 10 million cells, or any range in between these numbers. In some embodiments, the capsule contains densely packed cytochrome P450 producing cells, for example, about 10,000 cells/mm³, about 50,000 cells/mm³, about 100,000 cells/mm³, and the like. In some embodiments, the capsules contain monoclonal cells or cells of single subtype. In some embodiments, the capsules contain polyclonal cells or a mixture of subtypes.

In some embodiments, the amount of cytochrome P450 produced is about 0.1 pmol to about 100 pmol per capsule, about 0.1 pmol to about 10 pmol per capsule, or about 0.1 pmol to about 1 pmol per capsule.

In some embodiments, the capsule is semipermeable with pores that are large enough to allow prodrug molecules to pass through but small enough to prevent cells of the immune system from accessing the cells thereby significantly reducing an immune response to these cells. In some embodiments, the capsule membrane is porous and has a pore size from about 80 nm to about 150 nm, about 80 nm to about 120 nm, about 80 nm to about 100 nm, or about 50 nm to about 150 nm.

The capsules and the encapsulated cells are cultivated in a normal cell culture medium (the nature of which depends on the cell line encapsulated) at standard conditions of humidity, temperature and CO₂ concentration. After a suitable period in culture (normally not less than 1 hour and not exceeding 30 days), the capsules containing cells can be surgically implanted either directly, or by injection using a syringe into various areas.

In some embodiments, the method of treating a solid tumor involves administering the capsule containing cytochrome P450 producing cells at the tumor site, and administering a prodrug which is activated by cytochrome P450, wherein the prodrug is administered at least three or more cycles, and wherein each cycle comprises three consecutive daily administrations. The solid tumor may be pancreas cancer, liver cancer, ovarian cancer, sarcoma, and combinations thereof. Other non-limiting solid tumors include colorectal cancer, breast cancer, head and neck cancer, lung cancer, glioblastoma, melanoma, gastrointestinal cancer, prostate cancer, and cervical cancer.

The capsules may be administered by injection or by implantation surgically into the tumor or close to tumor. In some embodiments, the capsules may be directly injected into a blood vessel that supplies blood to the tumor and the surrounding organ.

In some embodiments, the method of treating a subject suffering from pancreas cancer comprises administering the capsule containing cytochrome P450 producing cells at the tumor site, and administering a prodrug which is activated by cytochrome P450, wherein the prodrug is administered at least three or more cycles, and wherein each cycle comprises three consecutive daily administrations. In some embodiments, the pancreas cancer may be unresectable, advanced, metastatic, or non-metastatic. In some embodiments, the subject suffering from pancreas cancer may not have undergone prior treatment, such as chemotherapy. In some embodiments, the method involves treating a subject suffering from pancreas cancer who is resistant to chemotherapy. In some embodiments, the subject suffering from pancreas cancer may have undergone prior treatment, such as first line of chemotherapy. Non-limiting chemotherapy agents include gemcitabine, taxanes, 5-fluorouracil, cisplatin, vincristine, vinblastine, altretamine, procarbazine, dacarbazine, temozolomide, etoposide, camptothecan analogs, doxorubicin, daunorubicin, epirubicin, mitoxantrone, idarubicin, or combinations thereof.

In some embodiments, a method of treating a subject suffering from pancreas cancer that no longer responds to or is resistant to combination of gemcitabine and nab-paclitaxel comprises administering the capsule containing cytochrome P450 producing cells at the tumor site, and administering a prodrug which is activated by cytochrome P450. In some embodiments, the subject suffering from pancreas cancer may have undergone gemcitabine and nab-paclitaxel therapy for 3-6 months. In some embodiments, the pancreas cancer is locally advanced, non-metastatic, and inoperable.

In some embodiments, the method of treating a subject suffering from pancreas cancer that no longer responds to or is resistant to combination of gemcitabine and nab-paclitaxel comprises administering the capsule containing cytochrome P450 producing cells at the tumor site, and administering a prodrug which is activated by cytochrome P450, wherein the prodrug is administered at least three or more cycles, and wherein each cycle comprises three consecutive daily administrations. In some embodiments, the subject suffering from pancreas cancer may have undergone gemcitabine and nab-paclitaxel combination therapy for 3-6 months. In some embodiments, the pancreas cancer is locally advanced, non-metastatic, and inoperable.

In some embodiments, capsules comprising cytochrome P450 producing cells are administered into blood vessels leading to the pancreas tumor using supraselective angiography with a catheter being inserted into a blood vessel in the groin, followed by low-dose ifosfamide administration given intravenously.

In some embodiments, the method further involves administering a prodrug. The prodrug is a prodrug activated by cytochrome P450 and is selected from oxazaphosphorines, dacarbazine, tegafur, flutamide, tamoxifen, duocarmycins, 2-aryl-benzothiazoles, AQ4N, PR-104, and derivatives thereof and combinations thereof.

In some embodiments, the prodrug is an oxazaphosphorine, such as cyclophosphamide, ifosfamide, trofosfamide, mafosfamide (NSC 345842), glufosfamide (D19575, beta-D-gluco sylisophosphoramide mustard), perfosfamide, S-(−)-bromofosfamide (CBM-11), NSC 612567 (aldophosphamide perhydrothiazine), NSC 613060 (aldophosphamide thiazolidine), and derivatives thereof and combinations thereof.

In some embodiments, the prodrug is a cyclophosphamide derivative of the formula (I):

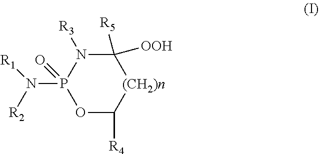

wherein R1 is selected from the group consisting of hydrogen and 2-chloroethyl;
R2 is 2-chloroethyl;
R3 is a member selected from the group consisting of hydrogen, lower alkyl of up to 6 carbon atoms, 2-chloroethyl and phenyl;
R4 and R5 are, independently, hydrogen and lower alkyl of up to 6 carbon atoms; and
n is 0 or 1, and pharmaceutical salts thereof.

The prodrug disclosed herein may be administered at least 3 cycles, and each cycle comprises administration for three consecutive days. In some embodiments, the prodrug is administered at a daily dose of about 1 gm/m²/day to about 2 gm/m²/day. In some embodiments, the period between two cycles of the prodrug administration is at least 18 days. For example, the capsule is administered on day 0, the first cycle of the prodrug is administered on days 2, 3, and 4; the second cycle of the prodrug is administered on days 23, 24, and 25; the third cycle of the prodrug is administered on days 44, 45, and 46, and so on.

Without wishing to be bound by theory, it is believed that in vivo cyclophosphamide is converted to a phosphoramide mustard that spontaneously cyclizes to an aziridinium DNA crosslinking agent. The release of the alkylating mustard involves cytochrome P450 hydroxylation adjacent to the ring nitrogen atom, reversible opening of the ring to give an aldophosphamide, and spontaneous decomposition of this intermediate to yield the phosphoramide mustard and acrolein. The activation of ifosfamide is similar to that of cyclophosphamide and is triggered by 4-hydroxylation of the drug by cytochrome P450, ring opening to give an aldehyde intermediate, elimination of acrolein to release the phosphamide and spontaneous formation of an aziridinium product that crosslinks DNA. This mechanism of action is the basis for the selectivity of ifosfamide cells that are rapidly dividing, such as tumor cells. Importantly, since the cells inside the capsules are not dividing they are not affected by the presence of the active 5-hydroxy-ifosfamide and are thus able to activate ifosfamide over long periods.

In some embodiments, the prodrug is cyclophosphamide or ifosfamide and is administered at a daily dose of about 1 gm/m²/day. In some embodiments, the subject is also administered sodium 2-mercatoethanesulfonate (MESNA) to prevent urotoxicity of cyclophosphamide and ifosfamide.

In some embodiments, the method involves administering about 200-500 capsules of cytochrome P450 producing cells at the tumor site and administering 3 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 4 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 5 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 6 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 7 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 8 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 9 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 10 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 11 cycles of the prodrug, wherein each cycle regimen involves administration for 3 consecutive days at a daily dose of about 1 gm/m²/day. The period between two subsequent cycles of the prodrug administration is at least 18 days.

In some embodiments, the method involves administering about 200-500 capsules of cytochrome P450 producing cells at the tumor site and administering 12 cycles of the prodrug, wherein each cycle regimen involves administration for 3 consecutive days at a daily dose of about 1 gm/m²/day. The period between two subsequent cycles of the prodrug administration is at least 18 days.

In some embodiments, the cytochrome P450 producing cells and the prodrug are administered together or sequentially. In some embodiments, the prodrug is administered systemically, locally, subcutaneously, intravenously, intramuscularly, intraperitoneally, transdermally, or orally. In some embodiments, the prodrug is administered as a sustained release composition.

In some embodiments, the capsules comprising cytochrome P450 producing cells may be administered multiple times along with the prodrug.

Formulations containing the prodrug can be solid dosage forms which include, but are not limited to, softgels, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. In some embodiments, a single dose may comprise one or more softgels, tablets, capsules, cachets, pellets, pills, or the like. Specific examples include, for example, a dose comprising 1, 2, 3, or 4 softgels, tablets, capsules, cachets, pellets, pills or the like.

In some embodiments, the prodrug can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Pharmaceutical compositions of the peptides/compounds also can comprise suitable solid or gel phase carriers or excipients. Examples of such carriers or excipients include, but are not limited to, calcium carbonate, calcium phosphate, various sugars, starches, cellulose derivatives, gelatin, and polymers such as, e.g., polyethylene glycols.

In addition to the formulations described above, the compositions of the prodrug can also be formulated as a depot preparation. Such long acting formulations can be administered by implantation (for example subcutaneously or intramuscularly) or by intramuscular injection.

Also, disclosed herein are methods to freeze and thaw HEK293 cells expressing cytochrome P450 without significant loss of cell viability. In some embodiments, the HEK293 cells (clone 22P1G) are grown in medium containing L-glutamine and frozen in serum free medium at −80° C. The cells are stored after freezing at −80° C. Upon unfreezing, the cells are revived in serum free medium that contains 10% fetal bovine serum (FBS). In some embodiments, the viability of the cells greatly improves when FBS is present. In some embodiments, the presence of FBS results in more rapid cell growth of the cells allowing them to reach their desired growth density for cell encapsulation within 5 days as compared to 18 days without FBS. In some embodiments, the cells are grown in FBS-free medium prior to encapsulation. After encapsulation to ultimately produce cell-filled capsules, the encapsulated HEK293 cytochrome P450 producing cells are grown inside the capsules, preferably in serum free medium. In some embodiments, the methods disclosed herein can produce capsules that have about 50% viable cells 12 weeks after encapsulation, about 60% viable cells 12 weeks after encapsulation, about 70% viable cells 8 weeks after encapsulation, about 80% viable cells 8 weeks after encapsulation, about 90% viable cells 8 weeks after encapsulation, about 95% viable cells 8 weeks after encapsulation, about 99% viable cells 8 weeks after encapsulation, and ranges between any two of these values.

Also, disclosed herein is a syringe with cytochrome P450 producing cells encapsulated in capsules. The syringe may be of any syringe shape known in the art; for example, a syringe having a body having an internal chamber, a proximal end through which a plunger assembly slides into and out of the chamber, and a distal end through which the contents of the chamber are discharged. A needle may be attached to the distal end to deliver the contents of the chamber.

In some embodiments, the syringe chamber includes a plurality of capsules, each capsule comprising a plurality of cytochrome P450 producing cells, and wherein capsule membrane is permeable to a prodrug. In some embodiments, the cytochrome P450 producing cells may be any mammalian cell, such as HEK293 cells. The cells may stably express cytochrome P450 gene under a regulatable promoter.

Further, the capsule may have a porous permeable membrane which allows nutrients to enter and waste products to leave. The pores also allow small molecules, such as the prodrug, to enter and leave the capsules. The pore size may range from about 80 nm to about 150 nm. In some embodiments, the capsule membrane is made from sulfate group containing polysaccharides, polysaccharide derivatives, sulfonate group containing synthetic polymers, polymers with quaternary ammonium groups, and combinations thereof.

In some embodiments, the capsule has an average diameter of about 0.001 mm to about 5 mm. Further, the capsule may include about 100-10 million HEK293 cells stably expressing cytochrome P450 gene.

In some embodiments, inner walls of the syringe are made of a cell non-adhesive material. Further, the syringe may also include a membrane that is permeable to oxygen and CO₂.

This invention and embodiments illustrating the method and materials used may be further understood by reference to the following non-limiting examples. The present invention is described in connection with the following examples which are set forth for the purposes of illustration only.

### EXAMPLES

### Example 1: Phase 1/2 Safety and Tolerability Study Details

This open-label, single site, non-randomized, historical control, Phase 1/2 study was conducted in patients >18 years of age with either a histological or cytological confirmed diagnosis of inoperable pancreas carcinoma (Stage II-IV). The 14 patients evaluated had not received previous therapy with ifosfamide, and were determined to have clinically measurable disease with an estimated life-expectancy of at least 6 months. The first patient visit occurred in July 1998, and the last patient visit was in September 1999. The study design included a 7 week Treatment Period (Days 0-49), which included instillation of the CypCaps on Day 0, and 2 cycles of chemotherapy on days 2-4 and 23-25, of 1 gm/m² ifosfamide along with 200 mg MESNA, followed by a 13-week Follow-up Period. Each CypCap (capsule) contained HEK 293 living cells (clone 22P1G) expressing the CYP2B1 gene encapsulated in polymeric sodium cellulose sulfate.

The primary objective was to evaluate the safety and tolerability of the angiographic instillation of encapsulated, genetically modified cells near a pancreas tumor. The secondary objective was to evaluate the clinical effects of the overall therapy as compared to untreated patients (historical data) with respect to response, survival, failure-free survival, and disease progression. Statistical analysis was performed on the data from the 14 patients who received CypCaps treatment. Comparison data included retrospective survival data from 36 patients with pancreas carcinoma treated by other means at the same facility from 1996 through 1998 that had not undergone tumor resection.

The majority of subjects in both populations were male (9 of 14 [64.3%] in the study population and 26 of 35 [74.3%]) in the historical population). The median age for both groups was 63 years, with a range of 49-77 years of age in the study population, and a range of 34-90 years of age in the historical population. All 14 (100%) patients in the treatment group were caucasian; however, race information is unknown for the historical group. For the treatment group, 12 (85.7%) patients were diagnosed as Stage IV and 2 (14.3%) were diagnosed as Stage III. For the historical group, 32 (91.4%) were diagnosed as Stage IV, 2 (5.7%) were Stage III, and 1 (2.9%) was Stage I.

The median survival time was 39 (range 6-52) weeks from histological diagnosis for the 14 patients in the treatment group as compared to the historical data median survival time of 20 weeks. At the 1 year post-clinical diagnosis timepoint, 5 (36%) patients in the treatment group and 4 (11%) patients in the control group were alive, indicating therapeutic benefit of the CypCaps instillation plus ifosfamide treatment (p=0.047). The median survival time (set to 52 weeks [e.g. censored data]) was significantly higher (p=0.008) in the treatment group (40 weeks) as compared to the controls (20 weeks).

CypCaps were instilled via angiography on study Day 0 for all 14 patients. A total of 13 patients received the anticipated 300 CypCaps, the remaining patient received 250 CypCaps due to limited space in the tumor artery. None of the patients experienced acute complaints during the angiography. Of the 14 completers, 11 patients received 2 cycles of treatment and 3 patients received only 1 cycle of ifosfamide chemotherapy (2 patients experienced poor health; 1 patient died).

All patients experienced at least 1 adverse event during the study, the majority of which were mild or moderate in severity (34.5% and 48.3%, respectively), and none were classified as related to CypCaps instillation. NCI toxicity ratings were collected, and any toxicity above Grade 3 was reported as an adverse event. Toxicities were to be evaluated during all visits at the hospital of the investigator. Of the 102 recordings after CypCaps instillation, 70 (68.6%) were Grade 1, 8 (7.8%) were Grade 2, and 24 (23.5%) were Grade 3 (pain: 22 events in 6 patients; nausea: 1 event in 1 patient; constipation: 1 event in 1 patient).

There were 3 deaths reported during this study (2 due to disease progression; 1 due to a pulmonary embolism). A total of 9 additional serious adverse events were reported by 7 patients during the study (1 event each of increased abdominal pain; stenosis of duodenum; cholangitis; septicemia due to cholangitis; elective chemo-embolization of liver metastases; cholestasis; bleeding of the gastrointestinal tract, anemia; jaundice, fever; aneurysma spurium right A; femoralis after angiography; and cholangitis due to choleostasis [drop-out]). None of these were deemed by the investigators to be related to the use of the treatment (CypCaps plus ifosfamide). In addition, 2 significant adverse events were reported by 2 patients (both were deterioration of health status). One patient did present with transient elevation of lipase activity, the reason for which was unclear, but this was self-correcting.

### Example 2: Phase 2 Efficacy and Safety Study Details

This open-label, multicenter, non-randomized, Phase 2 study was conducted in patients between 18 and 80 years of age with either a histological or cytological confirmed diagnosis of pancreas carcinoma (Stage II-IVA). The 13 patients evaluated had not received previous chemotherapy for pancreas cancer and were determined to have clinically measurable disease. The first patient visit occurred in November 1999, and the last patient visit was in December 2000. The study design included a Treatment Period, which included instillation of the CypCaps on Day 0, and 2 cycles of chemotherapy on Days 2-4 and 23-25, 2 gm/m² of ifosfamide along with 200 mg MESNA, followed by a Follow-up Period.

The primary objectives were to determine the tumor response rate (stable disease, partial response, and complete response) and to evaluate the clinical benefit (Karnofsky score, body weight, pain) of the CypCaps/ifosfamide treatment regimen in patients with inoperable pancreas cancer. The secondary objectives were to evaluate time to tumor progression, tumor response, duration of partial or complete response, length of time of symptom-free survival, and survival time of the patients. Additionally, safety and tolerability of the CypCaps/ifosfamide treatment were evaluated with special attention paid to the appearance of pancreatitis or immediate allergic reactions. Statistical analysis was performed on the data from the 13 patients who received the CypCaps/ifosfamide treatment.

All 13 patients were caucasian, 7 (53.8%) were male and 6 (46.2%) were female. The average age was 60.8±11.1 years (median was 59 years; range 43-78 years) and the average BMI was calculated to be 23.16±3.25 kg/m². All 13 patients suffered from inoperable pancreas carcinoma and 10 (76.9%) patients were diagnosed by central CT scans to have liver metastases. For the remaining 3 patients, there was no metastases in 1 patient, and for the remaining 2, the presence of metastatic lesions in the liver was unknown. A total of 12 (92.3%) patients were classified as having UICC (Union for International Cancer Control) Stage IVa and 1 (7.7%) patient was classified as Stage III pancreas cancer.

Diagnosis was confirmed histologically in 7 (53.8%) patients, cytologically in 5 (38.5%) patients, and information was missing for 1 patient. The average duration between diagnosis date and instillation of CypCaps was 41±22 days (range 14-82 days). Physical examination findings were normal with respect to the spleen, mucosa, and urinary tract for all patients. Abnormal findings were recorded in the abdomen (8 patients, mainly pain), cardiovascular system (3 patients), respiratory tract (3 patients), skin and eyes (2 patients), and liver and kidney (1 patient). Additionally, 2 patients had other reported discrepancies, with 1 patient suffering from major depression.

CypCaps were instilled via angiography on study Day 0 for all 13 patients. On average, 264±70 (median was 250) CypCaps were instilled prior to initiating treatment with ifosfamide. A total of 3 (23.1%) patients received the planned amount of 300 CypCaps. One (7.7%) patient received 450 CypCaps (part of the instillation solution rinsed into blood vessels that did not lead directly to the tumor), and 9 (69.2%) patients received less than 300 CypCaps (block of vascular flow after instillation of 280 CypCaps [1 patient], and a reduced diameter and number of tumor vessels with a risk of occlusion if more CypCaps were instilled [8 patients]).

A total of 12 patients received 2 cycles of treatment and 1 patient received only 1 cycle of ifosfamide chemotherapy (worsened health status). On average, the total dose of ifosfamide during the first cycle was 3538±328 mg and was 3481±295 mg during the second cycle. MESNA was injected prior to chemotherapy and again 4 and 8 hours later. During the first cycle the average dose was 3551±364 mg, and was 3518±343 mg during the second cycle.

A total of 3 (23.1%) patients died during the study period, 2 of which were in the 4-week follow-up period after early termination. A total of 6 (46.2%) patients died within 1 year after start of treatment and an additional 3 (23.1%) patients died within the second year. One patient was still alive at 61 weeks (last recorded follow-up) after study treatment. Overall, the median survival time was approximately 33 weeks after the initiation of treatment, and the 1-year survival rate was 23%. The lower survival rate, as compared to that seen in Phase 1/2 study, may be attributable to side effects of the higher dose of ifosfamide (2 gm/m²) administered in this Phase 2 study as compared to the previous Phase 1/2 study (1 gm/m²).

Overall, results of this Phase 2 study suggest that CypCaps plus ifosfamide is effective in the treatment of pancreas cancer; however, ifosfamide therapy at 2 gm/m² was toxic in the majority of the patients and was not significantly more effective in terms of antitumor activity than when the dose of ifosfamide was 1 gm/m² as used in the Phase 1/2 study.

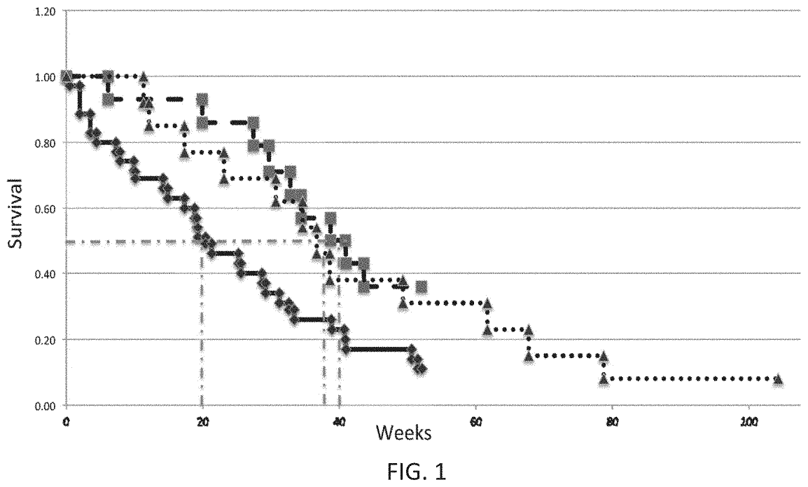

The data from the Kaplan-Meier survival curves for patients in each of the 2 studies are shown in FIG. 1. The median survival in the Phase 1/2 study was 39 weeks (ifosfamide dose was 1 gm/m²), whereas in the Phase 2 study, it was 33 weeks (ifosfamide dose was 2 gm/m²). Interestingly, the majority of the survival benefit was exhibited early in the study, with the patients eventually succumbing to the disease at a similar rate to the historic controls. This suggests that a prolongation of the survival benefit might be achieved if additional cycles of ifosfamide are given to the patients. The 1-year survival rate in the Phase 1/2 study was 36%, which was twice that of gemcitabine, the only drug approved by the FDA for the treatment of advanced pancreas cancer at the time the studies were conducted. By comparison, the 1-year survival for the Phase 2 study was 23%, which is possibly attributable to the side effects of the higher ifosfamide dose.

### Example 3: Installation of CypCaps and Administration of Prodrug for Longer Duration

CypCaps (300 capsules) will be instilled angiographically into a vessel leading to the pancreas tumor (Day 0). Ifosfamide (1 gm/m²/day) will be administered by intravenous infusion (over 30 min.) on days 2, 3, and 4, and every 3 weeks thereafter (i.e. on days 23, 24, and 25, and so on, until disease progression or lack of tolerability to the treatment occurs. When ifosfamide is administered, the uroprotector MESNA (at a 60% dose equivalent) will also be given (as 3 intravenous injections at (0, 4 and 8 hours) due to the urotoxicity characteristic of ifosfamide treatment. Ifosfamide/MESNA will be given for a total of 8 cycles if not limited by intolerability or tumor progression.

CypCaps will be implanted via a catheter and will be specifically delivered to the pancreas via the tumor vasculature under the guidance of supraselective angiography. Ifosfamide will be administered intravenously at low doses; hopefully, this will result in little to no systemic toxicity. Because the CypCaps are placed near the pancreas tumor, the activation of low dose ifosfamide should result in a significant anti-tumor effect (based on results from the previous Phase 1/2 and Phase 2 studies. A higher dose of the active form of ifosfamide will thus be achieved at the site of the tumor while systemic side effects from the ifosfamide treatment should be reduced than would otherwise be seen.

To illustrate the above, a prospective, multicenter, randomized, open-label Phase 2b study will be conducted in the U.S. to evaluate the efficacy and safety of CypCaps/ifosfamide vs. capecitabine/External Beam Radiation Therapy (EBRT), a standard of care, in patients with locally advanced, non-metastatic, inoperable pancreas cancer. The primary objective is to assess the comparative efficacy of the two combination treatments as determined by progression-free survival (PFS) measured from randomization to disease progression or death from any cause. Secondary objectives will compare the two treatments for (a) overall survival which will be evaluated as the time from randomization to death by any cause, (b) overall response rate which will be evaluated as the combined incidence of complete responses and partial responses in tumor size as defined by RECIST 1.1 criteria, (c) the proportion of patients whose pancreas tumors are converted from inoperable to operable, (d) the proportion of patients who have decreased levels of the tumor biomarker CA 19-9, and (e) patient functional outcomes as measured by differences in outcomes from baseline, during treatment and after treatment using the EORTC QLQ-C30 and the EORTC QLQ-PAN26 questionnaires.

In addition, the study will measure the safety and tolerability of the CypCaps/ifosfamide combination when ifosfamide is given multiple times during the study. Safety endpoints will be assessed as follows starting on day 0 through 28 days after the last dose of therapy; (a) overall frequency of adverse events, (b) vital signs, (c) 12-lead electrocardiographs, (d) serum antibody response in patients receiving the CypCaps/ifosfamide combination, (e) physical examinations and (f) clinical laboratory tests.

Only patients with locally advanced, non-metastatic, inoperable adenocarcinoma of the pancreas who have stable disease (determined by CT scans) after 4-6 cycles of chemotherapy with the combination of gemcitabine plus nab-paclitaxel (gem.nab) will be eligible for inclusion into the study. Rigorous inclusion and exclusion criteria will be used to screen the patients. Patients will randomized on a 1:1 basis into the two treatment groups (CypCaps/ifosfamide and capecitabine/EBRT). Randomization will be stratified by duration of previous chemotherapy; i.e. <6 cycles of gem/nab or 6 cycles of gem/nab. About 100 evaluable patients will be needed for the study.

The study will be composed of 6 periods. These are: (1) Screening and Randomization on days −20 through −14, (2) Scheduling and Preparation on days −13 to −1, (3) Treatment—for the CypCaps/ifosfamide group on days 0 to 169 and for the capecitabine/EBRT group on days 2 to day 43, (4) Observation—CypCaps/ifosfamide group from day 170 to progression/discontinuation and for the capecitabine/EBRT group from day 44 to progression or discontinuation and all patients will be evaluated for response according to RECIST 1.1 criteria which will take place every 8 weeks beginning on day 2 and a multidisciplinary team will determine whether a particular patients tumor is eligible for resection, (5) Early Termination—for patients who discontinue therapy after their first treatment assessment will be made about 30 days after their last study visit, and (6) Survival Follow-up—patient will be contacted directly, via caregiver, family member, etc. every 4 weeks after early termination until death by any cause or until study termination.

An interim analysis of the primary endpoint will be done when 50% of the total planned number (83) of progression-free survival events has occurred. The levels of significance for the interim and final analyses are alpha=0.00305 and 0.049, respectively. Enrollment into the study will continue during the interim analysis.

A statistical analysis of the data from the Phase 2b clinical study will be performed. The primary efficacy analysis will compare the progression-free survival in the CypCaps/ifosfamide and the capecitabine/EBRT treatment groups using a two-sided log rank test at the alpha=0.049 level of significance. The significance level for the final analysis will be adjusted because of the planned interim efficacy analysis. Secondary study endpoints defined as time-to-event variables will be analyzed using the log-rank test. Proportion endpoints will be analyzed using a chi-square test. Quantitative secondary endpoints will be analyzed using analysis of covariance models with the change from baseline as the dependent variable and treatment group and randomization stratification variables as factors, and the baseline value of the corresponding endpoint as a covariate. All secondary efficacy analyses will be conducted using two-sided tests at the alpha=0.05 level of significance. Safety parameters will be analyzed descriptively and presented in frequency tables (adverse events), shift tables (laboratory parameters), and summaries of changes from baseline over time (vital signs, pain and laboratory parameters).

The duration of study participation is to be as follows: (a) patients who receive CypCaps/ifosfamide are expected to participate for 24 weeks of treatment and then follow-up will occur until disease progression, discontinuation or death by any cause occurs (anticipated to be about 52 weeks), and (b) patients who receive capecitabine/EBRT therapy are expected to participate in the study for about 6 weeks of treatment with a follow-up period after treatment as for (a) above.

What is claimed is:

1. A method of treating a solid tumor in a subject in need thereof comprising:
administering at the site of the tumor in the subject a therapeutically effective amount of cytochrome P450 producing cells encapsulated in a capsule; and
administering to the subject a prodrug which is activated by cytochrome P450, wherein the prodrug is administered at least three or more cycles and wherein each cycle comprises administration for three consecutive days.

2. The method of claim 1, wherein the capsule is permeable to the prodrug and the prodrug is converted into an active moiety by cytochrome P450.

3. The method of claim 1, wherein the capsule is not permeable to cells of immune system.

4. The method of claim 1, wherein the prodrug is selected from oxazaphosphorines, dacarbazine, tegafur, flutamide, tamoxifen, duocarmycins, 2-aryl-benzothiazoles, AQ4N, PR-104, and derivatives thereof and combinations thereof.

5. The method of claim 1, wherein the solid tumor is selected from pancreas cancer, liver cancer, ovarian cancer, sarcoma, colorectal cancer, breast cancer, head and neck cancer, lung cancer, glioblastoma, melanoma, gastrointestinal cancer, prostate cancer, cervical cancer, and combinations thereof.

6. The method of claim 4, wherein the oxazaphosphorine is cyclophosphamide, ifosfamide, or a combination thereof, and is administered at a daily dose of about 1 gm/m²/day.

7. The method of claim 1, wherein period between two subsequent cycles of the prodrug administration is at least 18 days.

8. The method of claim 1, wherein the prodrug administration dose comprises 3 cycles to 12 cycles.

9. The method of claim 1, wherein the cytochrome P450 producing cells and the prodrug are administered to the subject together or sequentially.

10. The method of claim 1, wherein the cytochrome P450 producing cells are administered followed by administration of the prodrug after 24 hours.

11. The method of claim 1, wherein the prodrug is administered systemically, locally, subcutaneously, intravenously, intramuscularly, intraperitoneally, transdermally, or orally.

12. The method of claim 1, wherein the cytochrome P450 producing cells are HEK293 cells stably expressing cytochrome P450.

13. The method of claim 12, wherein the expression of cytochrome P450 is regulatable.

14. The method of claim 1, wherein the capsule comprises a capsule membrane comprising sulfate group containing polysaccharides, polysaccharide derivatives, sulfonate group containing synthetic polymers, polymers with quaternary ammonium groups, and combinations thereof.

15. The method of claim 14, wherein the capsule membrane comprises polymeric sodium cellulose sulfate.

16. The method of claim 14, wherein the capsule membrane is porous and has a pore size from about 80 nm to about 150 nm.

17. The method of claim 1, wherein the capsule comprises about 10² to about 10⁷ cytochrome P450 producing cells.

18. A HEK293 cell stably expressing cytochrome P450 2B1 isoform.

19. The HEK293 cells of claim 18, wherein the cells are resistant to freeze-thaw cycles.

20. A syringe comprising:
a plurality of capsules contained within the syringe, each capsule comprising a plurality of cytochrome P450 producing cells, and wherein capsule membrane is permeable to a prodrug.

21. The syringe of claim 20, wherein the cytochrome P450 producing cells are HEK293 cells.

22. The syringe of claim 20, wherein the capsule membrane comprises sulfate group containing polysaccharides, polysaccharide derivatives, sulfonate group containing synthetic polymers, polymers with quaternary ammonium groups, and combinations thereof.

23. The syringe of claim 22, wherein the capsule membrane comprises polymeric sodium cellulose sulfate.

24. The syringe of claim 20, wherein the capsule has an average diameter of about 0.001 mm to about 5 mm.

25. The syringe of claim 20, wherein the capsule membrane is porous and has a pore size from about 80 nm to about 150 nm.

26. The syringe of claim 20, wherein the capsule comprises about 100 cells to about 10 million HEK293 cells stably expressing a cytochrome P450 gene.

27. The syringe of claim 20, wherein the prodrug is selected from oxazaphosphorines, dacarbazine, tegafur, flutamide, tamoxifen, duocarmycins, 2-aryl-benzothiazoles, AQ4N, PR-104, and derivatives thereof and combination thereof.

28. The syringe of claim 20, wherein inner walls of the syringe are made of a cell non-adhesive material.

29. The syringe of claim 20, further comprising a membrane permeable to oxygen and CO₂.

30. A method of treating a subject suffering from pancreas cancer that is resistant to gemcitabine and nab-paclitaxel comprises administering at the tumor site a therapeutically effective amount of cytochrome P450 producing cells encapsulated in a capsule, and administering a prodrug which is activated by cytochrome P450.

31. The method of claim 30, wherein the prodrug is selected from oxazaphosphorines, dacarbazine, tegafur, flutamide, tamoxifen, duocarmycins, 2-aryl-benzothiazoles, AQ4N, PR-104, and derivatives thereof and combinations thereof.

32. The method of claim 31, wherein the oxazaphosphorine is cyclophosphamide, ifosfamide, or a combination thereof, and is administered at a daily dose of about 1 gm/m²/day.

33. A method of treating a subject suffering from pancreas cancer that is resistant to gemcitabine and nab-paclitaxel comprises administering at the tumor site a therapeutically effective amount of cytochrome P450 producing cells encapsulated in a capsule, and administering a prodrug which is activated by cytochrome P450, wherein the prodrug is administered at least three or more cycles, and wherein each cycle comprises three consecutive daily administrations.

34. The method of claim 33, wherein the prodrug is selected from oxazaphosphorines, dacarbazine, tegafur, flutamide, tamoxifen, duocarmycins, 2-aryl-benzothiazoles, AQ4N, PR-104, and derivatives thereof and combinations thereof.

35. The method of claim 34, wherein the oxazaphosphorine is cyclophosphamide, ifosfamide, or a combination thereof, and is administered at a daily dose of about 1 gm/m²/day.

36. The method of claim 33, wherein period between two subsequent cycles of the prodrug administration is at least 18 days.
